(19) United States
(12) Patent Application Publication
(10) Pub. No.: US 2017/0000430 A1
(43) Pub. Date: Jan. 5, 2017

(54) METHODS AND APPARATUS FOR JAW MOTION ANALYSIS

(71) Applicant: Carestream Health, Inc., Rochester, NY (US)

(72) Inventors: Yanbin Lu, Shanghai (CN); Qinran Chen, Shanghai (CN); Guijian Wang, Shanghai (CN); Wei Wang, Shanghai (CN)

(21) Appl. No.: 14/754,810

(22) Filed: Jun. 30, 2015

(51) Int. Cl. A61B 6/14; A61B 5/11; A61B 6/00; A61B 6/03

(52) U.S. Cl. CPC A61B 6/14; A61B 6/032; A61B 5/1128; A61B 6/5247

(57) ABSTRACT

An apparatus for imaging the head of a patient has a transport apparatus that orbits an x-ray source and detector about the head and acquires 2-D radiographic projection images. One or more head marker retaining elements hold markers in position relative to the skull. One or more jaw marker retaining elements hold markers in position relative to the jaw bone. At least one camera acquires a jaw motion study. A control logic processor in signal communication with the x-ray source, x-ray detector, and the camera is configured to reconstruct volume image content using the 2-D radiographic projection images acquired, to segment the jaw bone structure from the skull bone structure, to register the reconstructed volume image content, and to generate display content that shows jaw bone structure motion acquired from the set of reflectance images. A display in signal communication with the control logic processor displays the generated content.

### TECHNICAL FIELD

The disclosure relates generally to the field of volume imaging and more particularly to methods and apparatus for combining volume images that have been reconstructed from radiographic projection images of the human head of a patient with image content from motion studies of the patient.

### BACKGROUND

Radiological imaging is acknowledged to be of value for the dental practitioner, helping to identify various problems and to validate other measurements and observations related to the patient's teeth and supporting structures. Among x-ray systems with particular promise for improving dental care is the extra-oral imaging apparatus that is capable of obtaining one or more radiographic images in series and, where multiple images of the patient are acquired at different angles, combining these images to obtain a 3-D reconstruction showing the dentition of the jaw and other facial features for a patient. Various types of imaging apparatus have been proposed for providing volume image content of this type. In these types of systems, a radiation source and an imaging detector, maintained at a known distance (e.g., fixed or varying) from each other, synchronously revolve about the patient over a range of angles, taking a series of images by directing and detecting radiation that is directed through the patient at different angles of revolution. For example, a volume image that shows the shape and dimensions of the head and jaws structure can be obtained using computed tomography (CT), such as cone-beam computed tomography (CBCT), or other volume imaging method, including magnetic resonance imaging (MRI) or magnetic resonance tomography (MRT).

While 3-D radiographic imaging techniques can be used to generate volume images that accurately show internal structure and features, however, there are some limitations to the type of information that is available. One limitation relates to the static nature of the volume image content that is obtained. CBCT volume reconstruction requires the imaged subject to be stationary, so that 2-D radiographic image content that is used in reconstructing the 3-D volume information can be captured from a number of angles for combination. The patient must be still during imaging, so that points in image space can be accurately characterized in order to generate reliable voxel data. Only limited information useful for movement analysis for the jaws and related structure can be obtained due to CBCT imaging constraints. However, the capability to analyze and to visualize motion with the 3-D content can be very useful to help in diagnosing various conditions and in monitoring treatment results.

Motion analysis can be particularly useful for supporting the assessment and treatment of various conditions of the jaw, including diseases related to the mandibular joint and craniomandibular dysfunction, for example. Jaw motion analysis can also be of value in preparation of occlusal mouth-guards, dentures and other prosthetics, as well as in supporting aesthetically functional reconstruction, with or without tooth implants.

Conventional techniques for jaw motion analysis have included the dental pantograph that generates a graphical output representative of jaw movement. These methods are hampered by difficulties of setup and use, and provide only a relatively limited amount of information for characterizing jaw movement.

Solutions that have been proposed for providing jaw motion analysis include the use of various types of measurement sensors for determining the position of a jaw or facial feature in 3-D space. One exemplary system is described, for example, in U.S. Patent Application Publication No. 2003/0204150 by Brunner. To use this type of solution, it is necessary to attach and register various signal sensor and detector elements to the head and jaw of the patient, such as using head bands, bite structures, and other features. With these elements attached to the head and jaw, motion analysis data can be collected by having the patient move mouth and jaw in a fixed sequence of positions and recording the movement data. Then, once the movement sequence is complete, the motion information can be spatially correlated to the reconstructed 3-D volume, so that movement of the jaw and related structures can be modeled and assessed. The sensor and detector elements used for such a solution can include ultrasound emitters and receivers, cameras paired with LED markers or lasers, magnets and Hall effect sensors or pickup coils, and other types of motion detection apparatus.

The sensor and instrumentation detector attachment features currently in use or proposed for jaw motion analysis, however, can be somewhat bulky and awkward to use. Significant preparation time can be required for setting up the sensor/detector arrangement, including registering the instrumentation devices to the patient and to each other. The required instrumentation and harnessing can be costly and cumbersome and may make it difficult for the patient to move the mouth in a normal fashion, thus adversely affecting the motion data that is obtained.

Reference is also made to U.S. Pat. No. 4,836,778 to Baumrind et al., which shows a detector/sensor arrangement that employs infrared LEDs paired with photodiodes for measuring mandibular movement. U.S. Patent Application Publication No. 2013/0157218 by Brunner et al. shows another detector/sensor configuration for detecting jaw motion.

Video imaging of mandibular motion using three cameras in different positions about the patient has been described, for example, in U.S. Pat. No. 5,340,309 to Robertson.

International patent application publication WO 2013/175018 describes a method for generating a virtual jaw image.

Although various attempts have been made to provide sensing mechanisms that measure jaw motion, there is a need for improved methods that not only accurately profile jaw movement without significant cost or patient discomfort, but are also able to integrate this information with radiographic volume images from CBCT and related systems. The capability to relate jaw motion information with volume image content for the internal jaw structure can give the dental practitioner a useful tool for assessing a patient's condition, for providing an appropriate treatment plan, and for monitoring the status of a particular treatment approach.

### SUMMARY

It is an object of this application to advance the art of volume imaging and visualization used in medical and dental applications.

Another object of this application is to address, in whole or in part, at least the foregoing and other deficiencies in the related art.

It is another object of this application to provide, in whole or in part, at least the advantages described herein.

Method and/or apparatus embodiments of this application address the particular need for improved visualization and assessment of jaw motion, wherein internal structures obtained using CBCT and other radiographic volume imaging techniques can be correlated to motion data obtained from the patient. By combining volume image data with data relating to motion of the patient's jaw or other feature, method and/or apparatus embodiments of the present disclosure can help the medical or dental practitioner to more effectively characterize a patient's condition relative to jaw movement and to visualize the effects of a treatment procedure for improving or correcting particular movement-related conditions.

These objects are given only by way of illustrative example, and such objects may be exemplary of one or more embodiments of the invention. Other desirable objectives and advantages inherently achieved by the may occur or become apparent to those skilled in the art. The invention is defined by the appended claims.

According to one aspect of the disclosure, there is provided an apparatus for imaging the head of a patient, that can include a transport apparatus that moves an x-ray source and an x-ray detector in at least partial orbit about a head supporting position for the patient and configured for acquiring, at each of a plurality of angles about the supporting position, a 2-D radiographic projection image of the patient's head; one or more head marker retaining elements that hold a first plurality of markers in position relative to the skull of the patient; one or more jaw marker retaining elements that hold a second plurality of markers in position relative to the jaw bone of the patient; at least one camera that is disposed and energizable to acquire a jaw motion study comprising a set of a plurality of reflectance images from the head supporting position; a control logic processor that is in signal communication with the x-ray source, x-ray detector, and the at least one camera and that is configured by programmed instructions to reconstruct volume image content using the 2-D radiographic projection images acquired, to segment the jaw bone structure from the skull bone structure, to register the reconstructed volume image content to the first and second plurality of markers, and to generate animated display content according to jaw bone structure motion acquired from the acquired set of reflectance images; and a display that is in signal communication with the control logic processor and is energizable to display the generated animated display content.

According to one aspect of the disclosure, there is provided a method for recording movement of a jaw relative to a skull of a patient that can include orbiting an x-ray source and an x-ray detector in at least partial orbit about a head supporting position for the patient; acquiring, at each of a plurality of angles about the supporting position, a 2-D radiographic projection image of the patient's head; reconstructing a volume image from the acquired 2-D radiographic projection images; segmenting the jaw of the patient and the skull of the patient from the reconstructed volume image to form a reconstructed, segmented volume image; energizing a light source and recording a series of a plurality of reflectance images of the head during movement of the jaw of the patient; registering the recorded reflectance images to the reconstructed, segmented volume image according to a first set of markers that are coupled to a skull of the patient and a second set of markers that are coupled to the jaw of the patient; and generating and displaying an animation showing movement of the jaw bone relative to the skull according to the recorded series of reflectance images.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic diagram that shows an imaging apparatus for CBCT imaging of a patient.

FIGS. 2A, 2B, 2C, and 2D show top view schematics of transport apparatus rotating about the patient's head and head supporting position.

FIG. 3A is a top view schematic showing components of the imaging apparatus for obtaining radiographic and reflectance image content.

FIG. 3B is a top view schematic showing components of the imaging apparatus according to an alternate embodiment of the present disclosure.

FIG. 4A is a perspective view that shows imaging component disposition for reflectance imaging according to an embodiment of the present disclosure.

FIG. 4B is a perspective view that shows imaging component disposition for reflectance imaging according to an alternate embodiment of the present disclosure.

FIG. 4C is a perspective view that shows imaging component disposition for structured light image acquisition according to an embodiment of the present disclosure.

FIG. 5 is a schematic view that shows marker orientation relative to base axes for jaw motion imaging.

FIG. 6 is a schematic view showing marker positioning relative to the skull and jaw bone of the patient.

FIG. 7 is a perspective view showing features for marker positioning relative to the head of the patient.

FIG. 8 is a logic flow diagram that shows a sequence of procedures for displaying jaw motion.

FIGS. 9A through 9D show jaw marker movement during motion by the patient.

FIG. 10A is a perspective view that shows schematically how contour imaging is executed using a pattern of projected lines.

FIG. 10B shows a collection of lines used to form various types of patterns for surface contour imaging.

FIG. 11 is a view showing relation of markers to the patient's face for jaw motion detection.

### DETAILED DESCRIPTION OF THE EMBODIMENTS

The following is a detailed description of the preferred embodiments, reference being made to the drawings in which the same reference numerals identify the same elements of structure in each of the several figures.

Where they are used, the terms “first”, “second”, and so on, do not necessarily denote any ordinal or priority relation, but may be used for more clearly distinguishing one element or time interval from another.

As used herein, the term “energizable” relates to a device or set of components that perform an indicated function upon receiving power and, optionally, upon receiving an enabling signal. The opposite state of “energizable” is “disabled”.

The term “actuable” has its conventional meaning, relating to a device or component that is capable of effecting an action in response to a stimulus, such as in response to an electrical signal, for example.

The term “modality” is a term of art that refers to types of imaging. Modalities for an imaging system may be conventional x-ray, fluoroscopy, tomosynthesis, tomography, ultrasound, nuclear magnetic resonance (NMR), contour imaging, color reflectance imaging using reflected visible light, reflectance imaging using infrared light, or other types of imaging. The term “subject” refers to the patient who is being imaged and, in optical terms, can be considered equivalent to the “object” of the corresponding imaging system.

In the context of the present disclosure, the term “coupled” is intended to indicate a mechanical association, connection, relation, or linking, between two or more components, such that the disposition of one component affects the spatial disposition of a component to which it is coupled. For mechanical coupling, two components need not be in direct contact, but can be linked through one or more intermediary components or fields.

It will be understood that when an element is referred to as being “connected,” or “coupled,” to another element, it can be directly connected or coupled to the other element or intervening elements or magnetic fields may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The term “exemplary” indicates that the description is used as an example, rather than implying that it is an ideal.

The phrase “in signal communication” as used in the application means that two or more devices and/or components are capable of communicating with each other via signals that travel over some type of signal path. Signal communication may be wired or wireless. The signals may be communication, power, data, or energy signals which may communicate information, power, and/or energy from a first device and/or component to a second device and/or component along a signal path between the first device and/or component and second device and/or component. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the terms “pixel” and “voxel” may be used interchangeably to describe an individual digital image data element, that is, a single value representing a measured image signal intensity. Conventionally an individual digital image data element is referred to as a voxel for 3-Dimensional or volume images and a pixel for 2-Dimensional (2-D) images. Volume images, such as those from CT or CBCT apparatus, are formed by obtaining multiple 2-D images of pixels, taken at different relative angles, then combining the image data to form corresponding 3-D voxels. For the purposes of the description herein, the terms voxel and pixel can generally be considered equivalent, describing an image elemental datum that is capable of having a range of numerical values. Voxels and pixels have attributes of both spatial location and image data code value.

In the context of the present disclosure, the term “code value” refers to the value that is associated with each volume image data element or voxel in the reconstructed 3-D volume image. The code values for CT images are often, but not always, expressed in Hounsfield units (HU).

“Static” imaging relates to images of a subject without consideration for movement. “Patterned light” is used to indicate light that has a predetermined spatial pattern, such that the light has one or more features such as one or more discernable parallel lines, curves, a grid or checkerboard pattern, or other features having areas of light separated by areas without illumination. In the context of the present disclosure, the phrases “patterned light” and “structured light” are considered to be equivalent, both used to identify the light that is projected onto the head of the patient in order to derive contour image data.

In the context of the present disclosure, the terms “interference filter” and “dichroic filter” are considered to be synonymous.

In the context of the present disclosure, the terms “digital sensor” or “sensor panel” and “digital x-ray detector” or simply “digital detector” are considered to be equivalent. These describe the panel that obtains image data in a digital radiography system. The term “revolve” has its conventional meaning, describing movement along a curved path or orbit around a center point.

In the context of the present disclosure, the terms “viewer”, “operator”, and “user” are considered to be equivalent and refer to the viewing practitioner, technician, or other person who views and manipulates an x-ray image or a volume image that is formed from a combination of multiple x-ray images, on a display monitor. A “viewer instruction” or “operator command” can be obtained from explicit commands entered by the viewer or may be implicitly obtained or derived based on some other user action, such as making a collimator setting, for example. With respect to entries on an operator interface, such as an interface using a display monitor and keyboard, for example, the terms “command” and “instruction” may be used interchangeably to refer to an operator entry.

In the context of the present disclosure, a single projected line of light is considered a “one dimensional” pattern, since the line has an almost negligible width, such as when projected from a line laser, and has a length that is its predominant dimension. Two or more of such lines projected side by side, either simultaneously or in a scanned arrangement, provide a two-dimensional pattern.

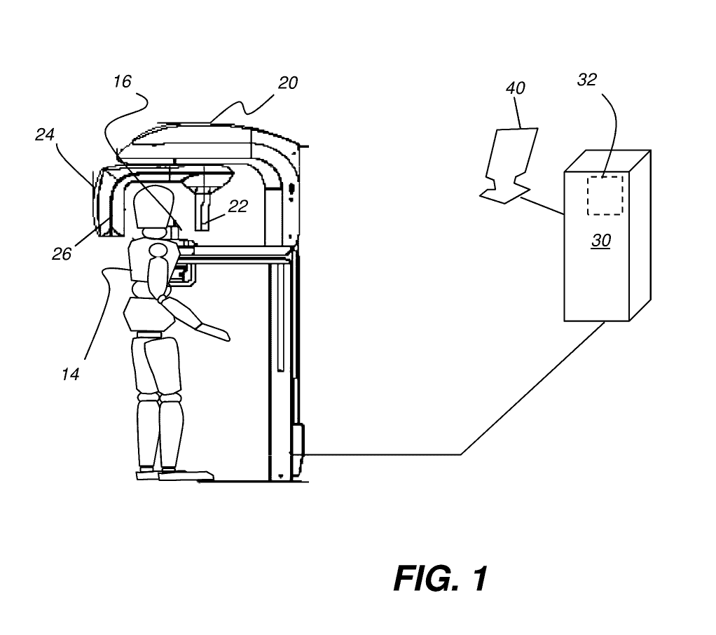

The schematic diagram of FIG. 1 shows an imaging apparatus 10 for acquiring, processing, and displaying a CBCT image of a patient 14. A transport apparatus 20 rotates a detector 22 and a generator apparatus 24 having an x-ray source 26 at least partially about a head supporting position 16 in order to acquire multiple 2-D projection images used for 3-D volume image reconstruction. A control logic processor 30 energizes x-ray source 26, detector 22, transport apparatus 20, and other imaging apparatus for reflectance image illumination and acquisition, as described in more detail subsequently, in order to obtain the image content needed for static 3-D imaging of the patient's face. To standardize patient 14 position at a suitable location for imaging, stabilize head position, and to provide a reference for orbiting the detector 22 and source 26 about the patient's head with suitable geometry for imaging, head supporting position 16 can include features such as a temple support and other supporting structures. Head supporting position 16 is a location at which the patient's head is located; however, there may or may not be features provided at head supporting position 16 for constraining movement of the head during imaging. Control logic processor 30 is in signal communication with a display 40 for entry of operator instructions and display of image results.

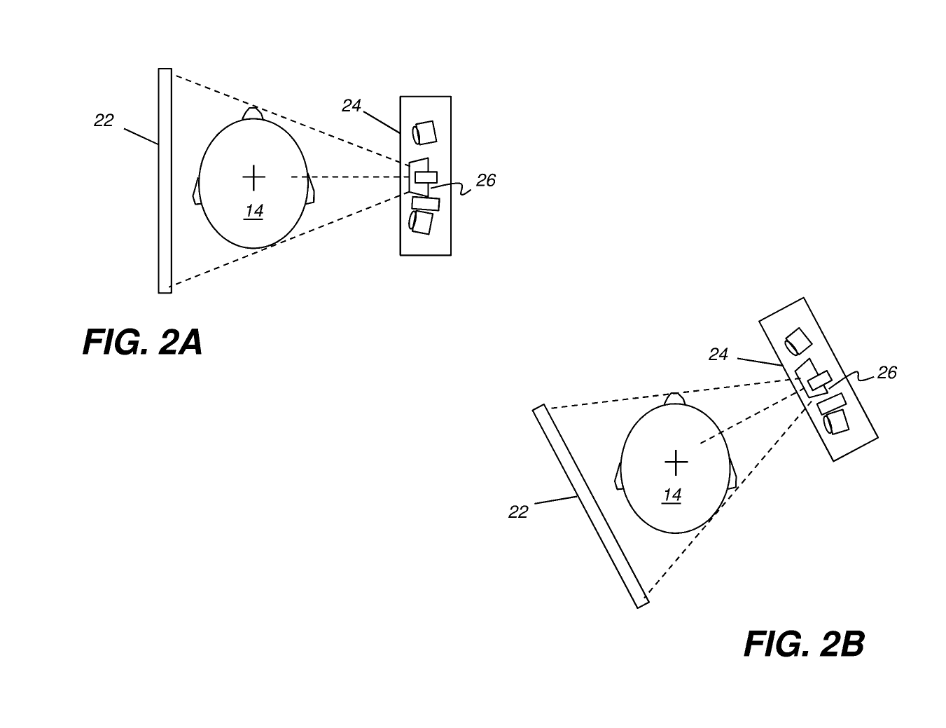

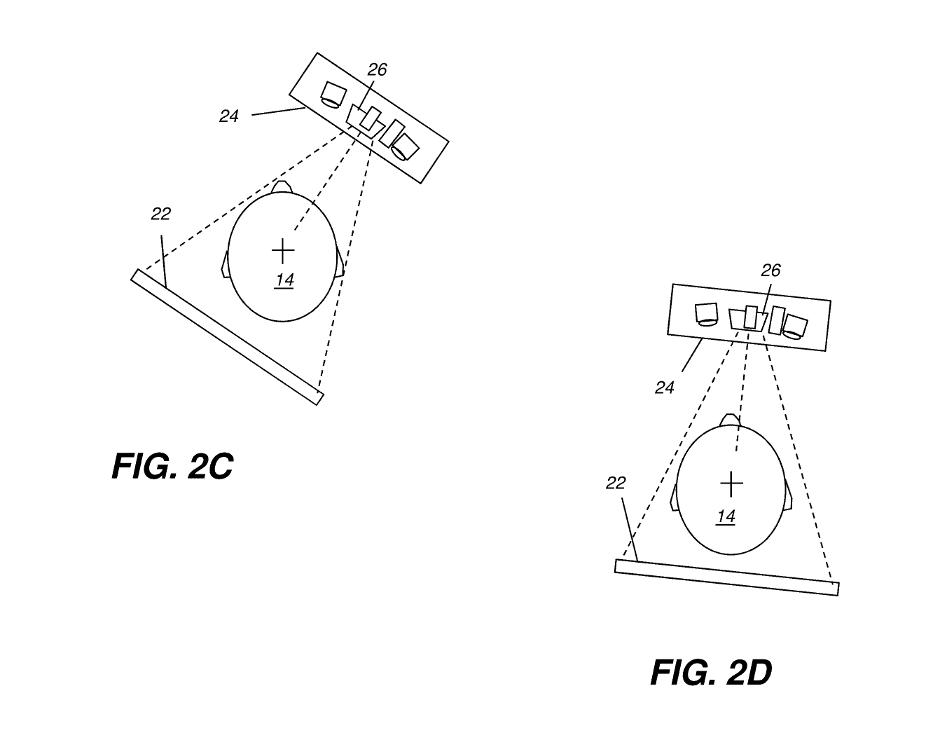

FIGS. 2A, 2B, 2C, and 2D show, for a few exemplary angles in top view schematic form, the action of transport apparatus 20 in orbiting generator apparatus 24 and detector 22 about the head of the patient 14 that is in head supporting position 16. The relative positions for generator apparatus 24 and detector 22 at four different representative angles are shown in FIGS. 2A-2D. At periodic angular increments during this rotation, x-ray source 26 is energized and detector 22 acquires the corresponding image content for the exposure at that angle. In the arrangement shown in FIG. 1, control logic processor 30 obtains the image data for each exposure, storing the 2-D projection image data for each exposure in a memory 32 for subsequent processing in order to generate the volume image content for display.

It should be noted that the orbit of generator apparatus 24 and detector 22 about the head of patient 14 is typically a circular orbit, but may alternately have a different shape (e.g., fixed or varying). For example, the orbit can be in the shape of a polygon or ellipse or some other shape. Different portions of the orbit can be used for the different types of image acquisition that are performed. In practice, radiographic imaging for CBCT reconstruction is performed over a range of angles. However, a small portion of the orbit can be used in some cases, such as imaging from a single angle for acquiring some types of reflectance images, to a substantial portion of the orbit, such as acquiring reflectance images at numerous incremental angles about the head, over a portion of the orbit that extends from one side of the head to the other.

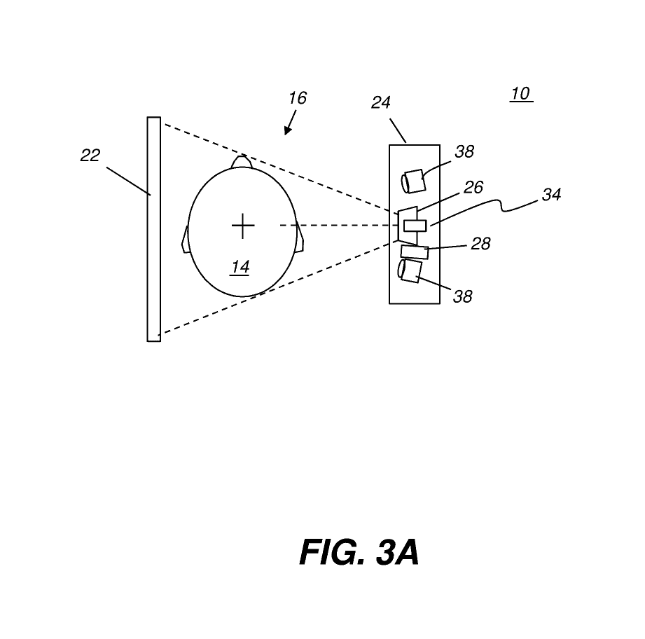

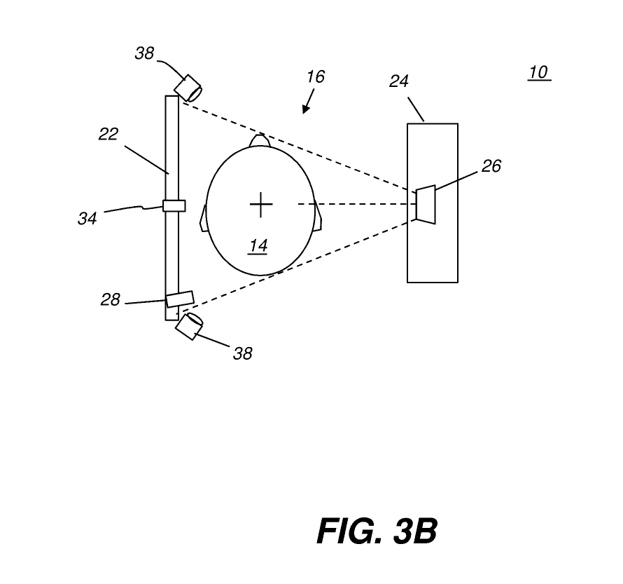

Method and/or apparatus embodiments of the present disclosure can adapt the basic CBCT imaging system described with reference to FIGS. 1-2D for the additional task of providing jaw motion analysis, using added components and with some changes to operational sequences. The top view schematic diagrams of FIGS. 3A and 3B show some of the components of imaging apparatus 10 for jaw motion sensing in more detail. Generator apparatus 24 houses x-ray source 26, as noted previously, and can include additional components for obtaining the desired reflectance image content for jaw motion analysis. Alternately, one or more of these additional imaging components can be provided adjacent to detector 22 as shown in FIG. 3B. Among these components can be an optional white light source 28, such as an LED or set of LEDs, that provides polychromatic or “white” light for obtaining reflectance images at two or more cameras 38. These reflectance images can record spatial positioning data on jaw movement that is combined with reconstructed volume image content from CBCT imaging from the same imaging apparatus, as described in more detail subsequently. Cameras 38 can be coupled to transport apparatus 20, as shown in subsequent figures or can be mounted separately from transport apparatus 20.

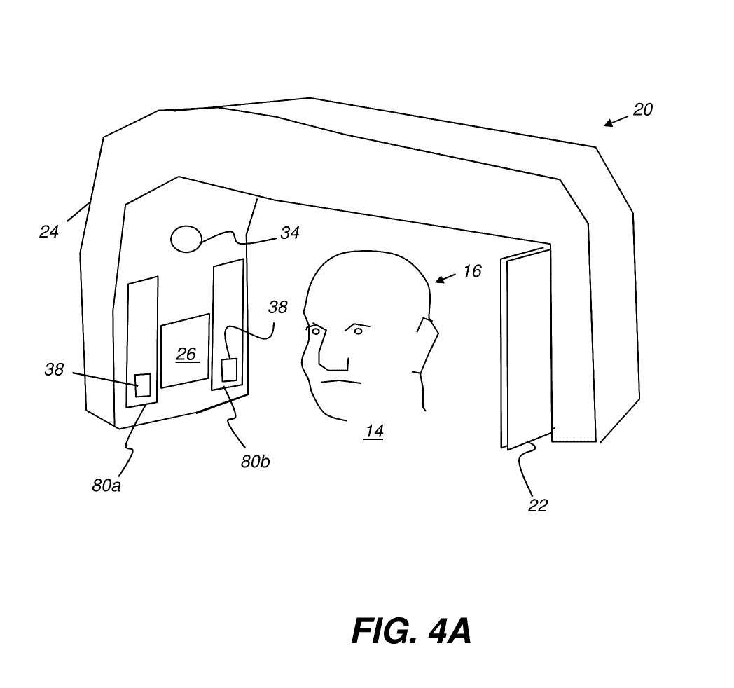

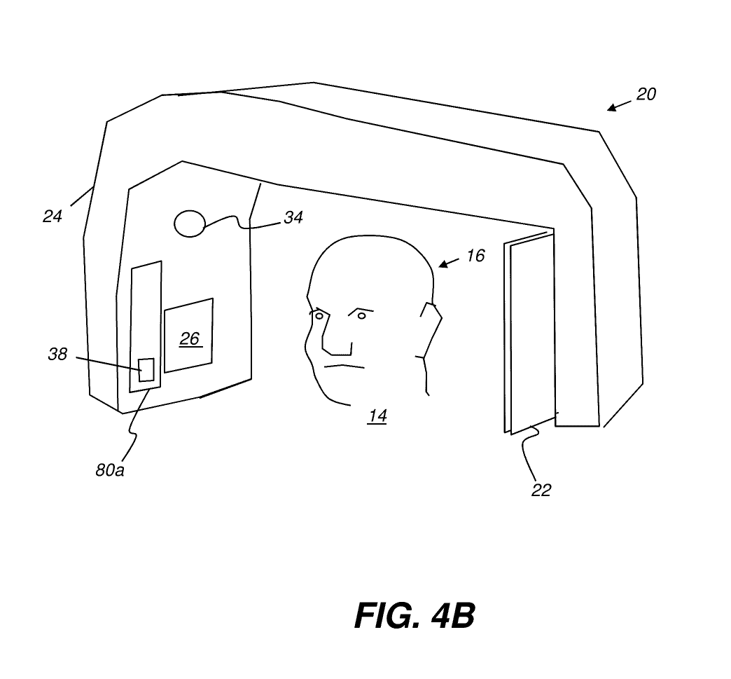

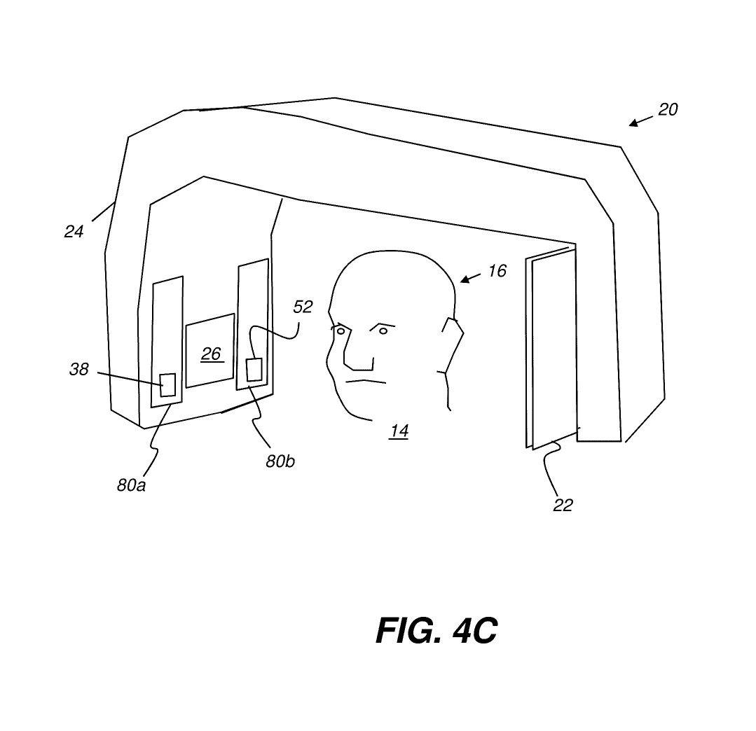

The perspective views of FIGS. 4A, 4B, and 4C show components used to obtain radiographic CBCT and reflectance image content for the face of patient 14 at head supporting position 16 according to embodiments of the present disclosure. Positions of x-ray source 26 and detector 22 within transport apparatus 20 for acquiring CBCT projection images are shown. FIG. 4A uses stereoscopic imaging and associated markers for motion tracking. Alternately, as shown in FIG. 4B, a single camera 38 is used and transport apparatus 20 provides the needed movement of the camera relative to the patient's head so that images can be obtained from multiple positions, as described in more detail subsequently. FIG. 4C uses yet another alternate approach, with structured light imaging and associated markers for motion tracking. It should also be noted that camera 38 can be mounted at any suitable position, either as part of transport apparatus 20, or separately positioned. Thus, for example, camera 38 could alternately be mounted alongside detector 22.

Referring first to FIG. 4A, in order to take advantage of image triangulation for acquiring depth information, two reflectance imaging assemblies 80a and 80b are provided within transport apparatus 20, with imaging assembly 80a imaging the head of patient 14 at a different angle from the image capture angle of imaging assembly 80b. The components that form imaging assembly 80a are similar to those that form imaging assembly 80b. Each imaging assembly 80a, 80b has a camera 38 and associated illumination components, as described in more detail subsequently.

In the alternate embodiment of FIG. 4B, a single imaging assembly 80a is used and transport apparatus 20 is actuated in order to obtain images from each of at least two positions, with the patient's head stationary. At each of the imaging positions, markers for the patient's skull and jaw are in the field of view of camera 38.

Referring to FIG. 4C, the arrangement of imaging assemblies 80a and 80b is different from that shown in FIG. 4A for stereoscopic triangulation in order to support the use of structured light imaging for jaw motion analysis. Imaging assembly 80b has a projector 52 for projecting structured light onto the patient's head. Imaging assembly 80a then has at least one camera 38 for obtaining images provided by the structured light. Triangularization techniques are used for determining the relative geometry of projector 52 and camera 38 positioning and angulation relative to the position of patient 14 at head supporting position 16. Multiple cameras 38 as well as multiple projectors 52 can be used for structured light imaging.

### Triangularization

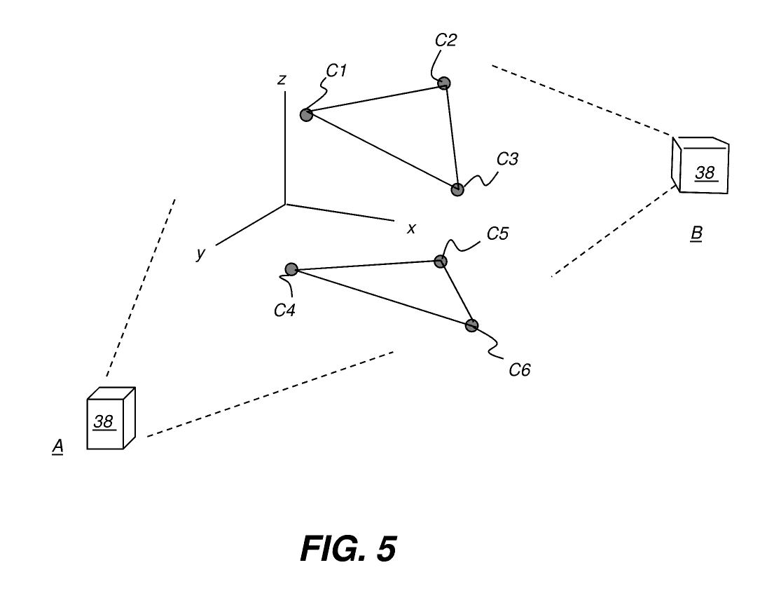

The schematic diagram of FIG. 5 shows one aspect of reflectance imaging that is useful for jaw motion analysis according to an embodiment of the present disclosure, using the component arrangement described with reference to FIG. 4A. Markers labeled C1 through C6 are visualized in 3-Dimensional space, each assigned standard Cartesian (x, y, z) coordinates, such as using the representative x,y,z axes shown. Markers C1, C2, and C3 are used to track skull position. Markers C4, C5, and C6 are used to track jaw movement. Cameras 38 are positioned at different angular locations for visual triangularization at positions A and B. Using techniques familiar to those skilled in the position-sensing arts, triangularization of the image capture apparatus and use of known geometry relative to the coordinates of the imaging system allows the cameras 38 to generate sufficient information for determining the relative coordinates of each of the markers C1-C6. As motion proceeds, the relative change in the positions of markers C1-C6 can be used to indicate motion patterns of associated, underlying skeletal structures. The use of three or more markers for the skull position and three or more markers for the jaw position can allow accurate detection and analysis of spatial translation and rotation of a rigid body relative to the 3-D coordinate system. Methods for generating a translation/rotation matrix based on relative movement of three nonlinear points using image triangularization are well known to those skilled in the motion analysis arts. In order to provide sufficient information for motion tracking, markers C1-C6 and cameras 38 are positioned so that the markers C1-C6 are preferably visible to both members of the pair of cameras 38 throughout the jaw motion study, as described in more detail subsequently.

In order to accurately identify marker Cl-C6 location and movement within the 3-D coordinate system, cameras 38 (FIGS. 4A, 5) can be paired, and each member of the pair synchronized to capture images of markers C1-C6 simultaneously. Multiple pairs of cameras 38 can be used, with each pair of cameras symmetrically disposed about the head of patient 14. Higher accuracy may be provided where two pairs of cameras 38 (that is, four cameras 38 total) are used. A first pair of cameras 38 may be used to capture images showing markers C1-C3; the other pair of cameras 38 would then be used to capture markers C4-C6 during jaw movement. This arrangement can be beneficial because marker C1-C6 positions may lie along the edge of the imaged field when only one pair of cameras is used. Distortion is higher along the edge of the imaged field.

Alternately, for the equipment configuration shown in the example of FIG. 4B, a single camera 38 is used to capture images of markers C1-C6 from different angular positions with respect to the patient's head. In the FIG. 4B arrangement, camera 38 is coupled to transport apparatus for providing images at two or more different angular positions.

For the structured light imaging system of FIG. 4C, triangulation is provided by the structured light optical system itself, so that projector 52 and camera 38 have a known geometric relationship that allows triangularization.

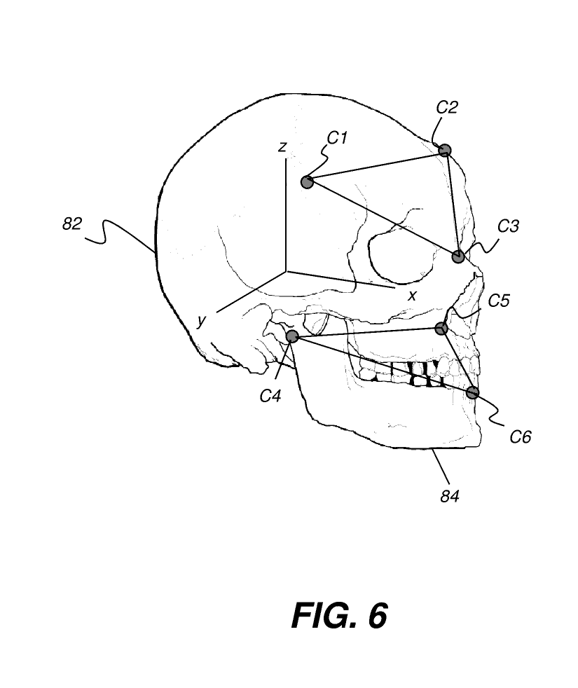

FIG. 6 shows, by way of example, how markers C1-C6 can be assigned to different positions along an object, such as a skull 82 and jaw 84. By identifying the relative coordinate locations of markers C1-C6, the corresponding positions of skull 82 and jaw 84 can be identified, such as using the triangularization imaging arrangement shown in FIG. 5. As shown by way of example in FIG. 6, markers C1, C2, and C3 are assigned to positions on skull 82. Markers C4, C5, and C6 are assigned to positions on jaw 84.

Capturing jaw motion for analysis can employ the basic reflectance image capture mechanism described with reference to FIGS. 5 and 6, but additional information is needed. Markers C1-C6 are not readily attached to the skull and jaw of the patient for imaging, but must be attached or coupled to the patient's head in some way that accurately associates each marker C1-C6 with a corresponding skeletal location. Thus, correlation of each marker C1-C6 location with a corresponding point on the jaw or skull is needed in order for jaw motion analysis to be able to utilize these reference points.

### Marker Arrangement and Attachment

In order to provide accurate motion tracking, markers C1-C6 must be positioned so that they correspond to appropriate positions along the skull and jaw and so that their own movement can be readily detected, over the full distance over which movement takes place. Three points in space define a plane. For skull marker C1-C3 placement, the first plane that is defined is non-parallel to the imaging plane of the camera 38 sensor. Similarly, for jaw marker C4-C6 placement, the second plane that is defined is also non-parallel to the imaging plane of the camera 38 sensor.

Each marker C1-C6 can be featured in some way that facilitates identification of its position and orientation. Identifying marker features can include color, reflective or retro-reflective portions, imprinted or formed shapes, markings such as geometric markings, alphanumeric labels, or symbols, etc. For structured light embodiments that use the component arrangement of FIG. 4C, markers C1-C6 that have distinctive 3-D information, such as by having different appearance from each other, can be advantageous. Because structured light techniques can discriminate between different marker shapes and sizes, the use of markers that differ in perceptible shape or size from each other can obviate the need for more sophisticated marking techniques. Specific markers can be assigned to particular positions along the skull or around the jaw. Alternately, color, patterning, numbers, or other encoding can distinguish each marker in appearance from the others.

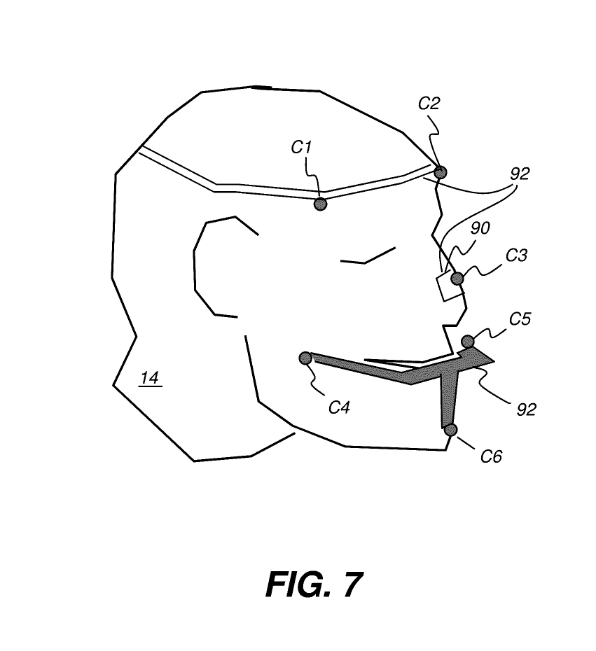

The perspective view of FIG. 7 shows positioning of markers C1-C6 according to an exemplary embodiment of the present disclosure. Skull markers C1 and C2 are arranged on a marker retainer element 92, such as a headband. Skull marker C3 is held in place by an adhesive 90, such as an adhesive strip that serves as marker retainer element 92. Jaw markers C4, C5, and C6 are held in place by a different type of marker retainer element 92, such as an element held in place by temporary adhesion to the lower teeth, for example. It can be appreciated that FIG. 7 shows one non-limiting possible arrangement for marker C1-C6 placement and retention to support jaw motion analysis; numerous other arrangements are possible using temporary adhesives, fixtures, bands, and other mechanisms.

In one exemplary embodiment, more than three markers are used for locating the skull position, and more than three markers are used for locating the jaw position, but at least three markers for each of the jaw position and skull position are arranged to be visible to tracking mechanisms (e.g. cameras) at any point in the respective scan or jaw motion analysis.

### Image Acquisition and Processing Sequence

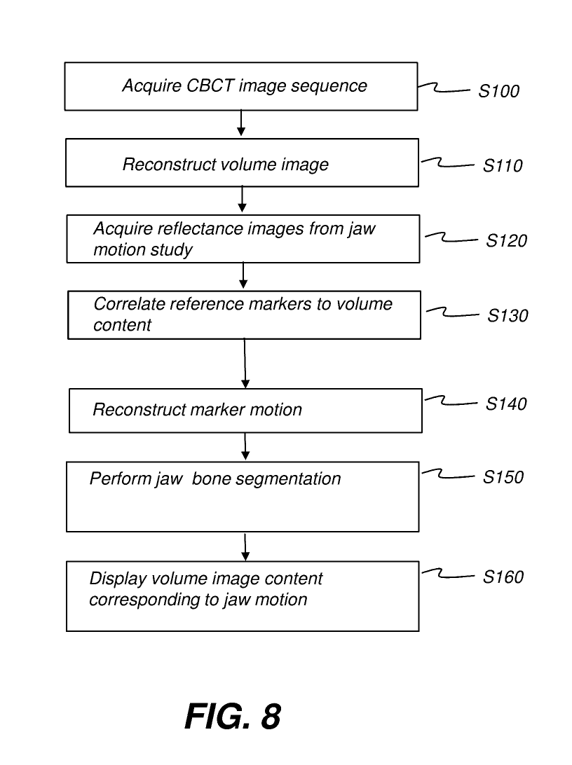

The logic flow diagram of FIG. 8 shows a sequence that can be used for image capture and processing to generate a visualization of jaw motion in three dimensions according to an embodiment of the present disclosure. While the order of individual steps may be varied, the sequence basically obtains the CBCT and reflectance image content that can be used or is needed for jaw motion analysis; to correlate the different types of image content to each other with regard to spatial locations; to process the volume image content using the obtained motion data; and/or to display jaw motion for the volume image content. The display that is generated can be in animated form, representing jaw movement relative to the skull as a type of motion picture having the appearance of a video stream. In alternative exemplary embodiments, jaw movements can be provide in static displays representative of an amount of movement of the jaw, of the skull, or of relative motion therebetween in 2D/3D graphics such as pictures, lists, charts, tables, vectors or the like.

Referring to FIG. 8, in a CBCT acquisition step S100 of imaging process 90, imaging apparatus 10 (FIG. 1) acquires the set of 2-D projection images that are needed for reconstruction of the 3-D volume image content. The 2-D images, for example, can be acquired at successive, incremental exposure angles as described with reference to FIGS. 2A-2D. The head of patient 14 is still; the x-ray source 26 and detector 22 are moved in at least partial orbit about the patient. A reconstruction step S110 is then executed on control logic processor 30, forming a volume image from the 2-D projection images acquired from step S100. Reconstruction step S110 can use some well known reconstruction algorithm for forming the volume image content, such as filtered back projection, Feldkamp-Davis-Kreis (FDK) reconstruction, or algorithmic reconstruction techniques, for example. The volume image content can be viewed from any of a number of angles and can be presented in the form of successive slices through the volume space, using presentation and display techniques that are familiar to those skilled in the volume image reconstruction arts.

Continuing with the FIG. 8 sequence, a jaw motion study S120 is executed. With the FIG. 4A or 4C apparatus embodiments, transport apparatus 20 (FIG. 1) is fixed at one angular position, so that the respective reflectance imaging assemblies 80a and, optionally 80b, are stationary. With the FIG. 4B embodiment, an initial set of images is obtained by moving camera 38 to capture images of the markers C1-C6 at at least two different positions about the head of the patient. The head of the patient may be partially restrained to constrain skull movement, but the jaw must be movable.

During jaw motion study S120, the patient follows instructions to execute a given sequence that can include jaw movement functions such as chewing, upwards/downwards and side-to-side motion, jutting motion, and retraction of the jaw, and other jaw movement. During jaw motion study S120, a succession of reflectance images is acquired from a single camera 38 or from two or more cameras 38 that are angled toward patient 14 and have markers C1-C6 in their field of view, as described previously with respect to FIG. 5. The acquired reflectance images can then be used to help visualize motion of the underlying jaw and skull structures in subsequent steps.

The FIG. 8 sequence can vary slightly for each of the different configurations of FIGS. 4A, 4B, and 4C. With the FIG. 4B embodiment, for example, an initial set of images is obtained, in which camera 38 captures images of the markers C1-C6 from at least different first and second positions (for example, rotating around the patient to locations where the markers are within the field of view of camera 38). Movement of the jaw in step S120 is then captured with the camera 38 held in place at a third position from which markers C-1-C6 are fully visible during all parts of jaw movement. This third position can be the same as the first or second position used earlier. Camera 38 is calibrated precisely at each of the positions, with the same intrinsic parameters (such as focus and depth of field) and different extrinsic parameters (such as camera position relative to coordinate axes). As one special case, if the camera rotates with the CBCT transport apparatus 20, the camera 38 can be calibrated in one position, with calibration results of other positions obtained using aligned CBCT geometric information that is known by the equipment configuration. For single-camera 38 use, as well as for the dual-camera configuration of FIG. 4A, at least three markers C1-C3 for the jaw and another three markers C4-C6 for the skull are used. Camera 38 can be coupled to either the generator apparatus 24 or the detector 22 side of transport assembly 20.

According to an alternate embodiment, the camera 38 can also move along an orbit or other trajectory during jaw movement if it is fully calibrated (both intrinsic and extrinsic parameters) over the whole trajectory.

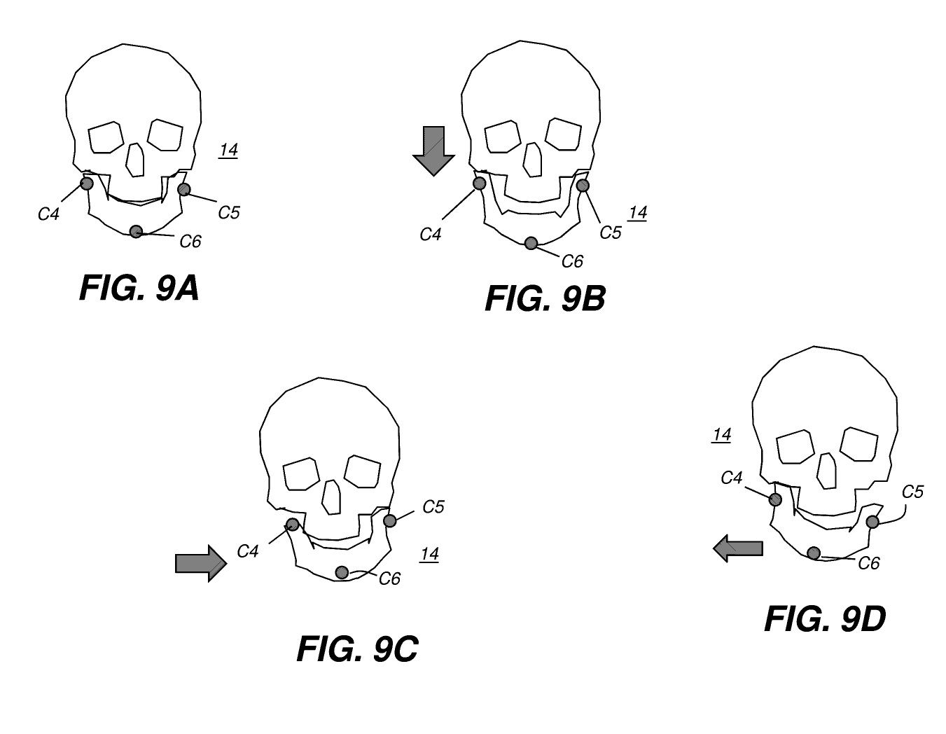

By way of illustration, the simplified schematic diagrams of FIGS. 9A-9D shows a portion of the markers C4, C5, C6 used in a motion study. Markers C4, C5, C6 each have a corresponding spatial location relative to jaw 84. During the study, patient 14 moves the jaw as instructed. Markers C4, C5, C6 move accordingly; this movement can be analyzed to help show jaw movement patterns.

A correlation step S130 associates the reference marker positions that are shown in acquired reflectance images with corresponding spatial positions in the reconstructed volume image content. This correlation can be executed in a number of ways, using methods well known to those skilled in the volume imaging arts. Correlation of reference marker C1-C6 positions can be performed by obtaining one or more radiographic images of the patient with the markers in place, for example, at a given angular position. Markers can alternately or additionally be located according to relative proximity to discernable structural features, for example.

Continuing with FIG. 8, a motion reconstruction step S140 records the sequences of marker motion for the different types of jaw movement, using reflectance images acquired during the motion study S120. The marker C1-C6 positions, correlated with the volume content in step S130 and recorded in step S140 can now serve to show movement of individual points of the jaw relative to the rest of the skull, but a segmentation step S150 is needed in order to isolate the jaw voxels from the skull structure, so that the jaw can be identified and visualized moving as a complete unit. With the jaw and skull identified as two separately movable structures, a display step S160 can be executed, using the marker motion and segmentation information for jaw movement. This allows jaw motion analysis by displaying the movement of the underlying mandibular bone structure relative to the skull. Display processing then shows the reconstructed CBCT volume image of the jaw as changing in translation and angle, while the volume image of the skull of patient 14 is at least substantially stationary. Display animation, using the changing jaw motion information from markers C4-C6, allows the display of step S160 to show motion of the jaw structure for analysis. The results from jaw motion analysis can also be stored for future use or transmitted between processors, such as for storage or display at a remote location.

Segmentation of the jaw can be provided using techniques familiar to those skilled in the image analysis arts and is well known in diagnostic imaging arts. Various segmentation methods could be used. According to an embodiment of the present disclosure, a template is used, coarsely outlining the jaw area, then refining and optimizing the segmentation result. A facial surface or 3-D tooth surface model can alternately be employed to help initiate or guide segmentation processing.

### Reflectance Imaging Approaches, Assemblies and Illumination Components

The reflectance images that are obtained can be used to show marker C1-C6 position and the overall surface of the patient's face during motion of the jaw. The components and methods that are used to acquire the reflectance images differ according to whether the imaging apparatus uses stereoscopic imaging as described with reference to FIG. 4A or structured light imaging, as described with reference to FIG. 4C.

Each reflectance imaging assembly 80a, 80b has at least one camera 38 and associated illumination components. As noted previously for stereoscopic imaging embodiments such as those described with reference to FIGS. 4A and 5, the cameras 38 for the reflectance imaging assembly 80a, 80b are provided in pairs, with suitable triangulation angles and synchronization of image capture timing. Reflectance illumination may be provided by a light source 34, which is coupled to transport apparatus 20 and may be an incandescent, Xenon, or halogen light source, or a solid-state light source such as a Light-Emitting Diode (LED) or laser source.

As noted previously for structured light embodiments as described with reference to FIG. 4C, projector 52 can provide a patterned light, also termed a structured light, that is then directed toward the head of patient 14 at head supporting position 16. Structured light can be provided by an optional light conditioning element such as a spatial light modulator, such as a Digital Light Processor (DLP) from Texas Instruments, Inc., Dallas Tex. or other micromirror array, or a liquid crystal device (LCD) array, for example. Alternately, the light conditioning element can be a grating or other device that forms a patterned or structured light when used in conjunction with light from a light source. The structured light pattern that is generated from projector 52 can be a one-dimensional (1-D) pattern, such as a single line at a time or a 2-D multiline pattern, or other type of 2-D pattern, including a grid or checkerboard pattern, for example. Light can alternately be provided by a scanner or other device that progressively forms a patterned light for projection onto a surface.

According to an embodiment of the present disclosure, projector 52 uses a near infrared (NIR) laser of Class 1, with a nominal emission wavelength of 780 nm, well outside the visible spectrum. Light from this type of light source can be projected onto the patient's face without awareness of the patient and without concern for energy levels that are considered to be perceptible or harmful at Class 1 emission levels. Infrared or near infrared light in the 700-900 nm spectral region appears to be particularly suitable for surface contour imaging of the head and face, taking advantage of the resolution and accuracy advantages offered by the laser, with minimal energy requirements. It can be appreciated that other types of lasers and light sources, at suitable power levels and wavelengths, can alternately be used.

Light source 34 is shown coupled to generator apparatus 24 in the embodiments shown in FIG. 4A. However, it should be noted that light source 34 can be coupled to transport apparatus 20 in some other position for orbital motion about the head supporting position 16.

In surface contour imaging, according to an embodiment of the present disclosure, projector 52 projects one 1-D line of light at a time onto the patient or, at most, not more than a few lines of light at a time, at a particular angle, and acquires an image of the line as reflected from the surface of the patient's face or head. This process is repeated, so that a succession of lines is obtained for processing as transport apparatus 20 moves to different angular positions. Other types of pattern can be projected, including irregularly shaped patterns or patterns having multiple lines. Projector 52 can be provided with an appropriate lens for forming a line, such as with a cylindrical lens or aspheric lens such as a Powell lens, for example. Additional optical components can be provided for shaping the laser output appropriately for contour imaging accuracy. The laser light can also be scanned across the face surface, such as using a rotating reflective scanner, for example. Scanning can be along the line or orthogonal to line direction.

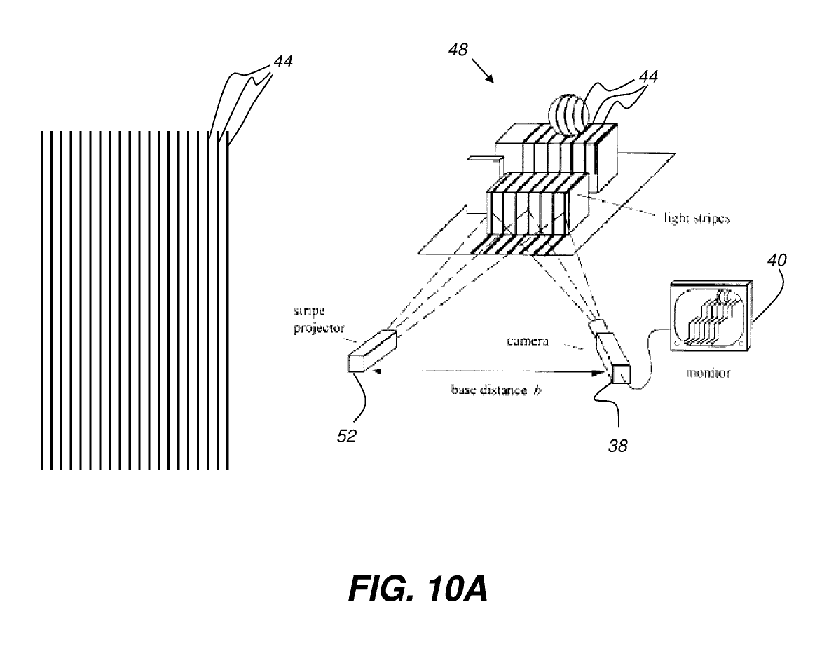

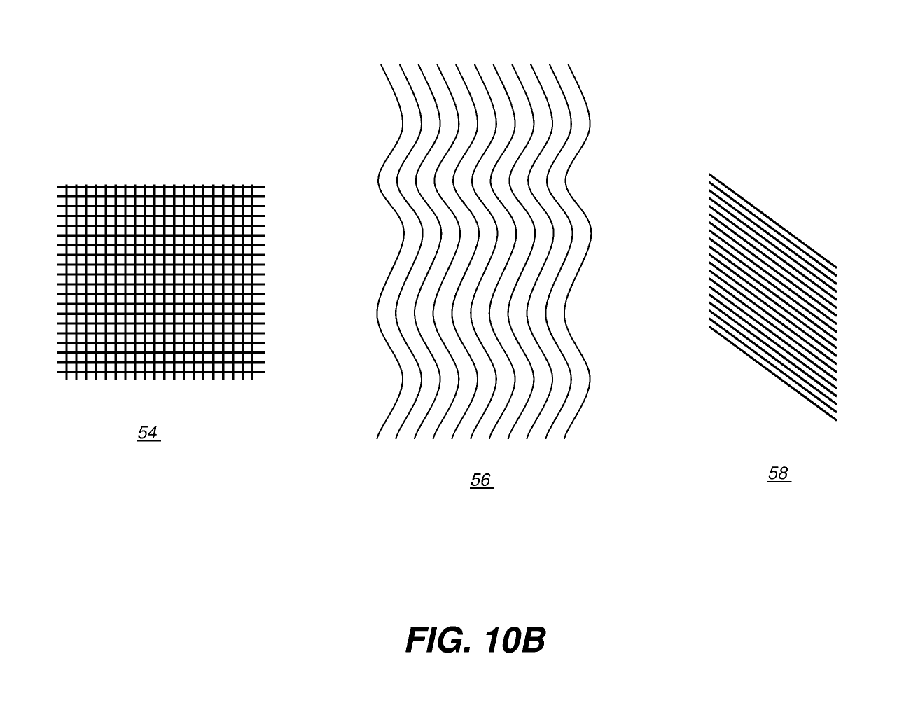

FIG. 10A shows, in simulated form, how surface contour imaging can be provided from a projector 52 using lines 44 individually projected from a laser source at different orbital angles toward a surface 48, represented by multiple geometric shapes. The combined line images, taken from different angles but registered to geometric coordinates of the imaging system, provide structured light pattern information. Triangulation principles are employed in order to interpret the projected light pattern and compute head and facial contour information from the detected line deviation. Lines 44 can be invisible to the patient and to the viewer.

The use of light outside the visible spectrum for forming lines 44 or other laser light pattern can be advantageous from a number of aspects. Lines 44 can be detected on a camera 38 that is sensitive to light at a particular wavelength, such as using one or more filters in the imaging light path.

FIG. 10B shows some of the other light patterns that can be projected onto the patient's face and used for surface contour imaging. Some of the 2-D light patterns that can be used include a grid 54 or checkerboard pattern, an arcuate pattern 56, and an oblique pattern 58, for example. Other possible patterns include patterns with scanned lines in different directions and patterns with lines of different thicknesses, different interline distances, and various types of encodings, for example. Sets of lines can be parallel or piece-wise parallel, such that adjacent segments of the projected line features extend in parallel directions.

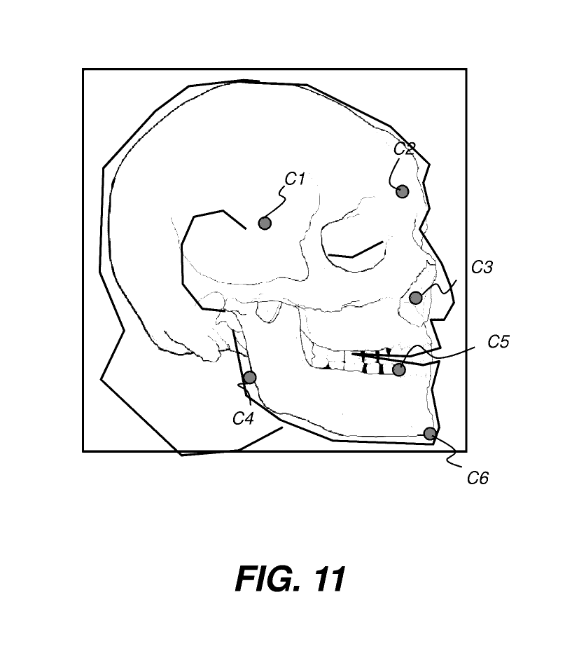

For display, the surface or contour information obtained from the reflectance imaging can have variable appearance when showing jaw motion. According to an embodiment of the present disclosure, the outline of the surface is shown, as shown in FIG. 11, optionally also showing the relative positions of markers C1-C6.

With respect to the camera(s) used to capture reflectance image content, it is useful to have calibration information that relates to the optical geometry of image acquisition. This type of intrinsic information includes data describing parameters such as focal length, imaging length (depth of field), image center, field of view, and related metrics. An initial calibration of the camera can be performed to identify optical characteristics, such as using a set of targets and executing an imaging sequence that captures image data from representative angles, for example.

Additional, extrinsic information relates the position of the imaging subject at head supporting position 16 (FIG. 1) to real-world coordinates for each type of imaging system that is used. Extrinsic geometry includes positional information on spatial location, angle, and related metrics for camera, lens, filter, and other optical components, relative to the head supporting position 16. Extrinsic geometry can be obtained by reconstructing a coarse point cloud using a set of color images from representative angles, then registering the coarse point cloud to a 3-D dense mesh obtained from contour image processing.

In the camera model at a position during jaw movement, logic processing that is executed by control logic processor 30 (FIG. 1) uses the changed marker positions to calculate the needed movement pattern for the corresponding features of the volume image. Methods that can be used for performing this calculation include using transformation matrices, for example, recomputing the relative position of each voxel of the volume image according to translation and rotation data obtained from marker movement.

Consistent with one embodiment, the present invention utilizes a computer program with stored instructions that control system functions for image acquisition and image data processing for image data that is stored and accessed from an electronic memory. As can be appreciated by those skilled in the image processing arts, a computer program of an embodiment of the present invention can be utilized by a suitable, general-purpose computer system, such as a personal computer or workstation that acts as an image processor, when provided with a suitable software program so that the processor operates to acquire, process, and display data as described herein. Many other types of computer systems architectures can be used to execute the computer program of the present invention, including an arrangement of networked processors, for example.

The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other network or communication medium. Those skilled in the image data processing arts will further readily recognize that the equivalent of such a computer program product may also be constructed in hardware.

It is noted that the term “memory”, equivalent to “computer-accessible memory” in the context of the present disclosure, can refer to any type of temporary or more enduring data storage workspace used for storing and operating upon image data and accessible to a computer system, including a database. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It is understood that the computer program product of the present invention may make use of various image manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present invention, are not specifically shown or described herein and may be selected from such algorithms, systems, hardware, components and elements known in the art.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim.

Exemplary embodiments according to the application can include various features described herein (individually or in combination).

While the invention has been illustrated with respect to one or more implementations, alterations and/or modifications can be made to the illustrated examples without departing from the spirit and scope of the appended claims. In addition, while a particular feature of the invention can have been disclosed with respect to one of several implementations, such feature can be combined with one or more other features of the other implementations as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated embodiment. Other embodiments of the invention will be apparent to those skilled in the art from consideration of the specification and practice of the invention disclosed herein. It is intended that the specification and examples be considered as exemplary only, with a true scope and spirit of the invention being indicated by the following claims, and all changes that come within the meaning and range of equivalents thereof are intended to be embraced therein.

What is claimed is:

1. An apparatus for imaging the head of a patient, comprising:
a transport apparatus that moves an x-ray source and an x-ray detector in at least partial orbit about a head supporting position for the patient and configured for acquiring, at each of a plurality of angles about the supporting position, a 2-D radiographic projection image of the patient's head;
one or more head marker retaining elements that hold a first plurality of markers in position relative to the skull of the patient;
one or more jaw marker retaining elements that hold a second plurality of markers in position relative to the jaw bone of the patient;
at least one camera that is disposed and energizable to acquire a jaw motion study comprising a set of a plurality of reflectance images from the head supporting position;
a control logic processor that is in signal communication with the x-ray source, x-ray detector, and the at least one camera and that is configured by programmed instructions to reconstruct volume image content using the 2-D radiographic projection images acquired, to segment the jaw bone structure from the skull bone structure, to register the reconstructed volume image content to the first and second plurality of markers, and to generate animated display content according to jaw bone structure motion acquired from the acquired set of reflectance images; and
a display that is in signal communication with the control logic processor and is energizable to display the generated animated display content.

2. The apparatus of claim 1 further comprising a solid-state light source that is coupled to the transport apparatus.

3. The apparatus of claim 2 wherein the light source emits polychromatic light.

4. The apparatus of claim 1 further comprising a projector that is energizable to direct a pattern of structured light toward the head supporting position.

5. The apparatus of claim 4 wherein the projector emits light in the near infrared or infrared spectral region.

6. The apparatus of claim 1 wherein the x-ray source and detector provide cone beam computed tomography imaging.

7. The apparatus of claim 1 wherein at least two of the markers differ from each other in shape or size.

8. The apparatus of claim 1 wherein the at least one camera acquires images from each of a first position and a second position relative to the head supporting position.

9. The apparatus of claim 1 wherein the at least one camera is coupled to the transport apparatus.

10. A method for recording movement of a jaw relative to a skull of a patient, the method executed at least in part by a computer and comprising:
orbiting an x-ray source and an x-ray detector in at least partial orbit about a head supporting position for the patient;
acquiring, at each of a plurality of angles about the supporting position, a 2-D radiographic projection image of the patient's head;
reconstructing a volume image from the acquired 2-D radiographic projection images;
segmenting the jaw of the patient and the skull of the patient from the reconstructed volume image to form a reconstructed, segmented volume image;
energizing a light source and recording a series of a plurality of reflectance images of the head during movement of the jaw of the patient;
registering the recorded reflectance images to the reconstructed, segmented volume image according to a first set of markers that are coupled to a skull of the patient and a second set of markers that are coupled to the jaw of the patient; and
generating and displaying an animation showing movement of the jaw bone relative to the skull according to the recorded series of reflectance images.

11. The method of claim 10 further comprising directing a structured light pattern toward the head from the energized light source.

12. The method of claim 10 further comprising recording a first reflectance image of the head from a first angular position relative to the head supporting position and recording a second reflectance image of the head from a second angular position relative to the head supporting position.

13. The method of claim 10 wherein recording the series of reflectance images comprises recording images that contain the first set of markers on a first camera and recording images that contain the second set of markers on a second camera.

14. The method of claim 10 wherein the markers differ from each other in shape, size, or appearance.
